SEQUENCE LISTING (4 sequences)

SEQ ID NO: 1 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 2 - DNA, 2776 bp, Homo sapiens
  misc_feature (2582)..(2582): n = c, a, t or g
cagttcaatgtttacacaatcctacagttctgctaggcttctatgatgctactattctgcatttgaatgagcaaatggatttaatgcattgtcagggagccggccaaagcttgagagctccttcctggctgggaggccccttggaatgtggcctgaaggtaagctggcagcgagcctgacatgctttcatctagtttcctcgcttccttccttttcctgcagttttcgcttcagagaaagcagaatccttaaaaataaccctcttagttcacatctgtggtcagtctgggcttaatggcaccccatcctccccatttgcgtcatttggtctcagcagtgaatggaaaaaagtgctcgtcctcacccccctgcttccctttcctacttcctggaaatccacaggatgctgcatttgctcagcagatttaacagcccacttatcactcatggaagatccctcctcctgcttgactccgccctctctccctctgcccgctttcaataagaggcagagacagcagccagaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccaggtaaggccccctcttcttctccttgaaccacattgtcttctctctgagttatcatggaccatccaagcagacgtggtacccacagtcttgctttaacgctacttttccaagataaggtgactcagaaaaggacaaggggtgagccccaaccacacagctgctgctcggcagagcctgaactagaattccagctgtgaacccaaatccagctccttccaggattcaggatccagctctgggaacacactcagcagttactcccccagctgcttccagcagagtttggggatcagggtaatcaaagagaagggtgggtgtgtaggctgtttccagacacgctggagacccagaatctggtctgtgcttcattcaccttagcttccagagaccggtgactctgcaggtaatgagtatcagggaaactcatgaccaggcatagctattcagagtctaaaaggaggctcatagtggggctcccagctgatcttccctggtgctgatcatctggattattggtccgtcttaatgacacttgtaggcattatctagctttaacagctcctccttctctctgtccattatcaatgttatataccccattttacagcataggaaactgagtcattgggtcaaagatcacattctagctctgaggtataggcagaagcactgggatttaatgagctctttctcttctcctgcctgccttttgttttttcctcatgactcttttctgctcttaagatcagaataatccagttcatcctaaaatgcttttctttgtggtttattttccagatgcaatcaatgccccagtcacctgctgctataacttcaccaataggaagatctcagtgcagaggctcgcgagctatagaagaatcaccagcagcaagtgtcccaaagaagctgtgatgtgagttcagcacaccaaccttccctggcctgaagttcttccttgtggagcaagggacaagcctcataaacctagagtcagagagtgcactatttaacttaatgtacaaaggttcccaatgggaaaactgaggcaccaagggaaaaagtgaaccccaacatcactctccacctgggtgcctattcagaacaccccaatttctttagcttgaagtcaggatggctccacctggacacctataggagcagtttgccctgggttccctccttccacctgcgtcctcctagtctccatggcagctcgcttttggtgcagaatgggctgcacttctagaccaaaactgcaaaggaacttcatctaactctgtctcctcccttccccacagcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaacctgacttccagtgttttcttgaaggaaattacaaagctgagagtatgagcttggtggtgacaaaggaacatgatttcagagggtggggcttacattttgaaggaatgggaaagtggattggcccnntntcttcctccactgggtggtctcctctgagtctccggtagaagaatctttatggcaggccagttaggcattaaagcaccacccttccagtcttcaacataagcagcccagagtccaatgaccctggtcacccatttgcaagagcccacccccatttcttttgctctcacgaccctgaccctgcatgcaattt

SEQ ID NO: 3 - protein, 76 aa, Homo sapiens
QPDAINAPVTCCYNFTNRKISVQRLASYRRITSSKCPKEAVIFKTIVAKEICADPKQKWVQDSMDHLDKQTQTPKT

SEQ ID NO: 4 - protein, 76 aa, Homo sapiens
  MOD_RES 1: Xaa is pyroglutamate
XPDAINAPVTCCYNFTNRKISVQRLASYRRITSSKCPKEAVIFKTIVAKEICADPKQKWVQDSMDHLDKQTQTPKT

(19) United States
(12) Patent Application Publication
(10) Pub. No.: US 2005/0148507 A1
(43) Pub. Date: Jul. 7, 2005

(54) Method for the production of an N-terminally modified chemotactic factor

(75) Inventors: Robert Wandl, Vienna (AT); Roman Necina, Vienna (AT); Henri Doods, Warthausen (DE); Martin Lenter, Ulm (DE); Randolph Seidler, Sandy Hook, CT (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/833,656

(22) Filed: Apr. 28, 2004

Related U.S. Application Data
(60) Provisional application No. 60/473,718, filed on May 27, 2003.

(30) Foreign Application Priority Data
May 2, 2003 (EP) 03010014.3

(51) Int. Cl. A61K 38/17; C07K 14/475

(52) U.S. Cl. 514/12; 530/350

(57) ABSTRACT

The invention relates to a process for preparing pyroGlu-MCP-1 from recombinantly produced Gln-MCP-1, wherein Gln-MCP-1 is incubated at a temperature in the range from 30° C. and 80° C. in a buffer solution with a salt concentration in the range from 10 mM to 160 mM and a pH in the range from 2 to 7.5, until at least 90% of the MCP-1 is present in the form of the pyroGlu-MCP-1.

The invention relates to a process for preparing pyroGlu-MCP-1 from recombinantly produced Gln-MCP-1 and compositions containing pyroGlu-MCP-1 preparations.

MCP-1 (monocyte chemoattractant protein-1; also known by the names: monocyte chemotactic and activating factor ‘MCAF’, macrophage chemotactic factor ‘MCF’, tumour necrosis factor stimulated gene-8 ‘TSG-8’, ‘HC-11’, smooth muscle cell chemotactic factor ‘SMC-CF’, lymphocyte derived chemotactic factor ‘LDCF’ as well as glioma derived chemotactic factor ‘GDCF’) is a member of the CC-chemokine family. Human MCP-1 protein was originally described in U.S. Pat. No. 5,714,578. It is synthesised under natural conditions in the body (natively) as a precursor protein 99 amino acids long, which is then processed to form a peptide with 76 amino acid groups. Mature human MCP-1 (hMCP-1) is a glycoprotein with a molecular weight of 14 kDa and is secreted by many types of cells, e.g. smooth vascular muscle cells and endothelial cells (Leonard and Yoshimura (1990), Immunology Today 11, 97-101). It contains two intramolecular disulphide bridges and is O-glycosylated and sialylated when expressed natively (J. Yan Ling et al. (1990), Journal of Biological Chemistry, 265,18318-18321).

The protein is a product of the JE gene of the chromosomal location 17q11.2-q21.1. This gene locus is also known as SCY A2 (small inducible cytokine A2). The human gene was first described in U.S. Pat. No. 5,212,073. The expression of this gene may be induced by a number of cytokines, such as e.g. tumour necrosis factor alpha, but also by immunoglobulin G, for example. The gene sequence and further information on the gene and the gene product are available in the NCBI Data bank under Accession Number M37719 (see Table 1).

TABLE 1
Human monocyte chemotactic protein gene, complete cds

LOCUS HUMMCHEMP 2776 bp DNA linear PRI 13-MAY-1994

| | |
|---|---|
| DEFINITION | Human monocyte chemotactic protein gene, complete cds. |
| ACCESSION | M37719 |
| VERSION | M37719.1 GI:187447 |
| KEYWORDS | monocyte chemotactic protein. |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (bases 1 to 2776) |
| AUTHORS | Shyy, Y.J., Li, Y.S. and Kolattukudy, P.E. |
| TITLE | Structure of human monocyte chemotactic protein gene and its |
| | regulation by TPA |
| JOURNAL | Biochem. Biophys. Res. Commun. 169 (2), 346-351 (1990) |
| MEDLINE | 90290466 |
| COMMENT | Original source text: Human DNA. |
| FEATURES | Location/Qualifiers |
| source | 1..2776 |
| | /organism=“homo sapiens” |
| | /db_xref=“taxon:9606” |
| gene | 598..2080 |
| | /gene=“SCYA2” |
| CDS | join(598..673, 1472..1589, 1975..2080) |
| | /gene=“SCYA2” |
| | /note=“monocyte chemotactic protein” |
| | /codon_start=1 |
| | /protein_id=“AAA18102.1” |
| | /db_xref=“GI:487124” |
| translation= | “MKVSAALLCLLLIAATFIPQGLAQPDAINAPVTCCYNFTNRKISVQRLA |
| | SYRRITSSKCPKEAVIFKTIVAKEICADPKQKWVQDSMDHLDKQTQTPKT” |
| exon | <598..673 |
| | /gene=“SCYA2” |
| | /note=“monocyte chemotactic protein” |
| | /number=1 |
| exon | 598..673 |
| | /gene=“SCYA2” |
| | /note=“monocyte chemotactic protein” |
| | /number=1 |
| intron | 674..1471 |
| | /gene=“SCYA2” |
| | /note=“monocyte chemotactic protein intron A” |
| exon | 1472..1589 |
| | /gene=“SCYA2” |
| | /note=“monocyte chemotactic protein” |
| | /number=2 |
| intron | 1590..1974 |
| | /gene=“SCYA2” |
| | /note=“monocyte chemotactic protein intron B” |
| exon | 1975..2080 |
| | /gene=“SCYA2” |
| | /note=“monocyte chemotactic protein” |
| | /number=3 |
| exon | 1975..>2080 |
| | /gene=“SCYA2” |
| | /note=“monocyte chemotactic protein” |
| | /number=3 |
| BASE COUNT | 700 a 727 c 565 g 781 t 3 others |
| ORIGIN | |
| 1 | cagttcaatg tttacacaat cctacagttc tgctaggctt ctatgatgct actattctgc |
| 61 | atttgaatga gcaaatggat ttaatgcatt gtcagggagc cggccaaagc ttgagagctc |
| 121 | cttcctggct gggaggcccc ttggaatgtg gcctgaaggt aagctggcag cgagcctgac |
| 181 | atgctttcat ctagtttcct cgcttccttc cttttcctgc agttttcgct tcagagaaag |
| 241 | cagaatcctt aaaaataacc ctcttagttc acatctgtgg tcagtctggg cttaatggca |
| 301 | ccccatcctc cccatttgcg tcatttggtc tcagcagtga atggaaaaaa gtgctcgtcc |
| 361 | tcacccccct gcttcccttt cctacttcct ggaaatccac aggatgctgc atttgctcag |
| 421 | cagatttaac agcccactta tcactcatgg aagatccctc ctcctgcttg actccgccct |
| 481 | ctctccctct gcccgctttc aataagaggc agagacagca gccagaggaa ccgagaggct |
| 541 | gagactaacc cagaaacatc caattctcaa actgaagctc gcactctcgc ctccagcatg |
| 601 | aaagtctctg ccgcccttct gtgcctgctg ctcatagcag ccaccttcat tccccaaggg |
| 661 | ctcgctcagc caggtaaggc cccctcttct tctccttgaa ccacattgtc ttctctctga |
| 721 | gttatcatgg accatccaag cagacgtggt acccacagtc ttgctttaac gctacttttc |
| 781 | caagataagg tgactcagaa aaggacaagg ggtgagcccc aaccacacag ctgctgctcg |
| 841 | gcagagcctg aactagaatt ccagctgtga acccaaatcc agctccttcc aggattcagg |
| 901 | atccagctct gggaacacac tcagcagtta ctcccccagc tgcttccagc agagtttggg |
| 961 | gatcagggta atcaaagaga agggtgggtg tgtaggctgt ttccagacac gctggagacc |
| 1021 | cagaatctgg tctgtgcttc attcacctta gcttccagag accggtgact ctgcaggtaa |
| 1081 | tgagtatcag ggaaactcat gaccaggoat agctattcag agtctaaaag gaggctcata |
| 1141 | gtggggctcc cagctgatct tccctggtgc tgatcatctg gattattggt ccgtcttaat |
| 1201 | gacacttgta ggcattatct agctttaaca gctcctcctt ctctctgtcc attatcaatg |
| 1261 | ttatataccc cattttacag cataggaaac tgagtcattg ggtcaaagat cacattctag |
| 1321 | ctctgaggta taggcagaag cactgggatt taatgagctc tttctcttct cctgcctgcc |
| 1381 | ttttgttttt tcctcatgac tcttttctgc tcttaagatc agaataatcc agttcatcct |
| 1441 | aaaatgcttt tctttgtggt ttattttcca gatgcaatca atgccccagt cacctgctgc |
| 1501 | tataacttca ccaataggaa gatctcagtg cagaggctcg cgagctatag aagaatcacc |
| 1561 | agcagcaagt gtcccaaaga agctgtgatg tgagttcagc acaccaacct tccctggcct |
| 1621 | gaagttcttc cttgtggagc aagggacaag cctcataaac ctagagtcag agagtgcact |
| 1681 | atttaactta atgtacaaag gttcccaatg ggaaaactga ggcaccaagg gaaaaagtga |
| 1741 | accccaacat cactctccac ctgggtgcct attcagaaca ccccaatttc tttagcttga |
| 1801 | agtcaggatg gctccacctg gacacctata ggagcagttt gccctgggtt ccctccttcc |
| 1861 | acctgcgtcc tcctagtctc catggcagct cgcttttggt gcagaatggg ctgcacttct |
| 1921 | agaccaaaac tgcaaaggaa cttcatctaa ctctgtctcc tcccttcccc acagcttcaa |
| 1981 | gaccattgtg gccaaggaga tctgtgctga ccccaagcag aagtgggttc aggattccat |
| 2041 | ggaccacctg gacaagcaaa cccaaactcc gaagacttga acactcactc cacaacccaa |
| 2101 | gaatctgcag ctaacttatt ttcccctagc tttccccaga caccctgttt tattttatta |
| 2161 | taatgaattt tgtttgttga tgtgaaacat tatgccttaa gtaatgttaa ttcttattta |
| 2221 | agttattgat gttttaagtt tatctttcat ggtactagtg ttttttagat acagagactt |
| 2281 | ggggaaattg cttttcctct tgaaccacag ttctacccct gggatgtttt gagggtcttt |
| 2341 | gcaagaatca ttaatacaaa gaattttttt taacattcca atgcattgct aaaatattat |
| 2401 | tgtggaaatg aatattttgt aactattaca ccaaataaat atatttttgt acaaaacctg |
| 2461 | acttccagtg ttttcttgaa ggaaattaca aagctgagag tatgagcttg gtggtgacaa |
| 2521 | aggaacatga tttcagaggg tggggcttac attttgaagg aatgggaaag tggattggcc |
| 2581 | cnntntcttc ctccactggg tggtctcctc tgagtctccg gtagaagaat ctttatggca |
| 2641 | ggccagttag gcattaaagc accacccttc cagtcttcaa cataagcagc ccagagtcca |
| 2701 | atgaccctgg tcacccattt gcaagagccc acccccattt cttttgctct cacgaccctg |
| 2761 | accctgcatg caattt |
| // | |

It has already been explained in the above-mentioned U.S. Pat. No. 5,714,578 that in the case of an MCP-1 protein isolated from a native source the N terminus is blocked. Only later was it discovered that this is due to a post-translational modification in which the glutamine exposed after the cleaving of the leader sequence at the N terminus of the mature protein is cyclised, losing an NH₃ molecule, to form a pyroglutamate group.

The identification of the open reading frame coding for MCP-1 in U.S. Pat. No. 5,212,073 allowed recombinant expression of the protein. An MCP-1 produced by the recombinant method, e.g. in E. coli, does not have the typical N- or O-glycosylation pattern of the native MCP-1. An MCP-1 preparation prepared in this way also does not oppose Edman decomposition in the same way as a preparation obtained from native material, i.e. it contains unblocked N termini (glutamines). In cellular assays based on the chemotactic effect on macrophages, originally no difference could be found between the biological activity of native MCP-1 preparations and those obtained by the recombinant method.

Under physiological conditions MCP-1 acts as an agonist to the beta-chemokine receptors CCR2 and CCR4, both of which are expressed mainly on monocytes and are also found both on basophiles and on T- and B-lymphocytes. MCP-1 induces monocyte chemotaxis even at subnanomolar concentrations. The receptors CCR2 and CCR4 are G-protein-coupled seven-transmembrane domain receptors which lead to the activation of monocytes and increased adhesion of integrins. This process ultimately results in the docking of monocytes to endothelial cells and the subsequent departure of the monocytes from the vascular system.

WO 98/44953 discloses the influence of MCP-1 on arteriogenesis, i.e. the growth of collateral arteries and/or other arteries from existing arteriolar connections. On the basis of this finding it was proposed in the above-mentioned International Patent Application to use MCP-1 for therapeutic purposes, namely for the treatment of vascular occlusive diseases which may be alleviated by stimulating the formation of new blood vessels. Such vascular occlusive diseases are particularly coronary artery disease (CAD), peripheral arterial occlusive disease (PAOD), cerebral and mesenterial arterial occlusive diseases, etc. The application also proposed the use of MCP-1-neutralising agents such as e.g. anti-MCP-1-antibody for preventing new vascular formation in order to combat tumour growth, in particular, which is reliant on a sufficient blood supply and hence adequate vascularisation of the tumour tissue.

In order to be able to use the MCP-1 protein as explained above in a therapeutic approach for promoting the vascularisation of tissue, large enough quantities of this protein have to be made available with sufficient purity for pharmaceutical purposes. Human MCP-1 was originally obtained in native form from cultures of the human glioma cell line U105MG or from human mononuclear leukocytes of peripheral blood. Protein intended for therapeutic purposes cannot be isolated from these sources because the production of large enough amounts would only be possible at unacceptably high cost in terms of labour and/or materials: it would not be possible to obtain human leukocytes in the required quantity. The glioma cells could indeed be replicated in virtually unrestricted amounts, but are unsuitable for cultivation in biotechnological fermenters and additionally require foetal calf serum for their cultivation, for example, with all the attendant problems.

Instead of the native expression of MCP-1 it is therefore an inviting prospect to prepare MCP-1 by the recombinant method. In fact recombinant human MCP-1 is already commercially available, particularly from Messrs R&D Systems (Catalogue no. 279-MC) and Peprotech (Catalogue no. 300-04; see Table 2; the catalogue numbers quoted are those applicable in January 2003). According to the product specification of the commercially available recombinant MCP-1 preparations the MCP-1 protein present therein contains the amino acid glutamine (Q) at the N terminus. The product description also indicates that this protein preparation is highly sensitive, particularly in the reconstituted liquid form (recommended max storage: 1 week at 4° C.). In fact, experiments by the inventor (internal prior art) showed that when the conventional MCP-1 protein preparations are stored in dissolved form the—biologically active—protein preparation appears to show signs of contamination by breakdown products very rapidly, particularly at temperatures above 4° C. (appearance of secondary bands in analytical HPLC chromatographs).

TABLE 2
(extract from the Internet Website of Messrs Peprotech
http://www.peprotech.com/content/details.htm?results=1&prod=1392):
Recombinant Human MCAF (Human MCP-1)

| | |
|---|---|
| Description: | Human monocyte chemotactic protein-1 (MCP-1) also known as |
| | macrophage/monocyte chemotactic and activating factor (MCAF) is an 8.6 |
| | kDa protein containing 76 amino acid residues. It plays an important role in the |
| | inflammatory response of blood monocytes and tissue macrophages. |
| Catalog #: | 300-04 |
| Source: | E.coli |
| Formulation: | The sterile filtered solution was lyophilized with no additives. |
| Stability: | The lyophilized protein is stable for a few weeks at room temperature, but best |
| | stored at −20° C. Reconstituted human MCAF should be stored in working |
| | aliquots at −20° C. |
| Purity: | Greater than 99% by SDS-PAGE and HPLC analyses. Endotoxin level is less |
| | than 0.1 ng per μg (1EU/μg). |
| Reconstitution: | We recommend a quick spin followed by reconstitution in water to a |
| | concentration of 0.1-1.0 mg/ml. This solution can then be diluted into other |
| | aqueous buffers and stored at 4° C. for 1 week or −20° C. for future use. |
| Biological | Determined by its ability to chemoattract human monocytes using a |
| Activity: | concentration range of 5.0-20.0 ng/ml. |
| AA Sequence: | QPDAINAPVT CCYNFTNRKI SVQRLASYRR ITSSKCPKEA |
| | VIFKTIVAKE ICADPKQKWV QDSMDHLDKQ TQTPKT |
| Country of | USA |
| Origin: | |

A conventional method of recombinantly producing human MCP-1 protein is by the temperature-induced expression of the protein as a fusion protein, purification of the inclusion bodies formed subsequently, dissolving and refolding of the protein and subsequent enzymatic release of the hMCP-1 protein from the fusion protein.

In conventional processes for the recombinant preparation of human MCP-1 protein the problem arises that a protein which is N-terminally shortened by one or more amino acids is often obtained as a by-product. These by-products may behave as antagonists to MCP-1 receptors in biological systems.

In Van Coillie et al. (1998), Biochemistry 37: 12672, 12673 a.E. a process is described in connection with experiments on the influence of N-terminal modifications on the biological activity of MCP₋₂, wherein an MCP-2 preparation obtained by the recombinant method is incubated in 0.01 M Na₂HPO₄, pH 8.0, for 24 hours at 37° C. MCP-2 has 62% homology with MCP-1 at the level of the amino acid sequence and also differs from it to the extent that N-terminally unmodified MCP-1 is biologically active, unlike MCP-2 (cf. the line “Biological Activity” in Table 2). The authors did not determine the extent of the conversion of the N-terminal glutamine groups into a pyroglutamate group which takes place under the conditions mentioned above. The inventors have found (internal prior art) that the application of comparable conditions to MCP-1 in any case leads to only partial cyclisation of the N-terminal amino acid (cf. Table 3, Comparison test 2).

One aim of the invention therefore, in the light of the foregoing discussion, is to provide a process by which an MCP-1-preparation (MCP-1 composition) may be prepared, which (a) is suitable for therapeutic purposes in view of its biological activity and (b) has advantages in terms of the drug licensing procedures, which in the case of biopharmaceuticals contain requirements which are extremely difficult to comply with, e.g. with regard to the purity and reproducibility of production of the pharmaceutical composition, and in particular (c) has an excellent shelf life. Another related aim is to provide compositions or preparations which contain MCP-1 produced by the recombinant method and are suitable for the purposes mentioned above or have the aforementioned advantages.

These aims are achieved by the processes and compositions containing MCP-1 recited in the claims.

Thus according to the invention a process for preparing pyroGlu-MCP-1 from recombinantly produced Gln-MCP-1 is provided wherein Gln-MCP-1 is incubated at a temperature in the range from 30° C. and 80° C., preferably in the range from 30° C. and 70° C. and more preferably in the range from 35° C. to 60° C., in a buffer solution which has a salt concentration in the range from 10 mM to 160 mM, preferably in the range from 10 mM to 100 mM, and which has a pH in the range from 2 to 7.5, preferably 3.5 to 7.5, more preferably 3.5 to 6.5, more preferably 5 to 6.5, more preferably 5.5 to 6.5 and more preferably has a pH of about 6. The incubation is carried out until at least 90%, preferably at least 95% and more preferably at least 96% of the MCP-1 contained in the incubating buffer solution is present in the form of the pyroGlu-MCP-1.

By “MCP-1” is meant, within the scope of this disclosure, the MCP-1 protein (without preprosequence), with an N-terminal glutamine group (“Gln-MCP-1”; cf. the amino acid sequence shown in Table 1 and Table 2) or with an N terminus which has already been converted/cyclised into the pyroglutamate group (“pyroGlu-MCP-1”), depending on the context. It is clear, however, that modifications of the MCP-1-protein which do not affect its biological function (esp. the monocyte-attractant activity or the effect on the CCR receptor) and do not alter its structure so that the reaction parameters described above no longer produce the desired result (i.e. the protein can no longer be converted into a pyroGlu-Variant by the process according to the invention), do not depart from the scope of protection. Thus, the process according to the invention is naturally also applicable to an MCP-1 in which a conservative amino acid exchange, e.g. serine to threonine or leucine to isoleucine, has taken place, provided that this affects neither the biological function or activity of the resulting protein nor the convertibility of the N-terminal glutamine into pyroglutamate according to the above process. The process according to the invention can thus also be applied to MCP-1 proteins of other mammals such as e.g. rats, mice, guinea pigs, rabbits and ferrets (and several others).

The buffer solution in which the process described above is carried out is preferably a phosphate-buffered (sodium and/or potassium phosphate-buffered) aqueous solution of low or physiological molarity, namely in the range from 10 to 160 mM, 10 to 80 mM, 10 to 50 mM, 20 to 40 mM or around 20 or 40 mM. If longer incubation times are accepted, according to one particular embodiment of the invention the work may also be done at physiological molarities, such as e.g. a saline concentration of around 150 mM, this molarity preferably being achieved by the use of a phosphate-buffered saline solution or “PBS”. The disadvantage of the longer incubation period—and the attendant risk of increasing amounts of breakdown, secondary or oxidation products—is made up for in this particular embodiment by the advantage of being able to obtain the protein preparation straight away in the form of a solution with a physiological salt concentration.

The buffer solution in which the modification step is carried out may also contain, for example, a (mild) detergent, an antioxidant, a preservative, a complexing agent, stabilisers, antimicrobial reagents, etc.

The incubation temperature is selected in the range from 30° C. to 80° C., i.e. above ambient temperature. At lower temperatures the conversion step proceeds very slowly. In order to achieve virtually total conversion the incubation period would have to be increased substantially to more than a week. This would lead to stand times which are unacceptable in the biotechnological process and would also substantially increase the risk of other forms of contamination of the protein solution (with breakdown or oxidation products, bacteria, viruses or other pathogens). On the other hand, although increasing the incubation temperature to above 80° C. does indeed greatly speed up the reaction of conversion, it also results in an increased formation of undesirable by-products and breakdown products (cf. Examples 1 and 2). If stand times of up to 6 days are acceptable, an incubation temperature of 35 to 40° C. is particularly preferred. In cases where incubation should be significantly shorter if possible, the incubation may also be carried out in the range from e.g. 50 to 60, 70 or 80° C. Temperatures between 40 and 50° C. will naturally also produce the desired results.

The pH of the incubating buffer solution should be in the range from 2 and 7.5, i.e. in the neutral to acidic range. It is preferably in the range from 3.5 to 7.5, more preferably in the range from 3.5 to 6.5, more preferably in the range from 5 to 6.5, more preferably in the range from 5.5 to 6.5 and more preferably a pH of about 6 is selected.

With a suitable choice of the above parameters, e.g. as described in the Examples, the pyroGlu-MCP-1 variant may be obtained with a purity of 90% in any case and possibly higher, e.g. 95%, 96% or 98%, this percentage indicating the amount of pyroGlu-MCP-1 (determined e.g. as the area under the curve in an HPLC elution profile) based on the amount of total MCP-1 present in the solution (i.e. including any remaining amount of Gln-MCP-1 and possible oxidation products and other by-products or breakdown products).

According to another embodiment of the invention a process for preparing a pyroGlu-MCP-1 preparation is provided which comprises at least the following steps:
preparing a Gln-MCP-1 preparation by the recombinant method by expression of a gene construct coding for MCP-1 in a host cell,
optionally concentrating and/or purifying the Gln-MCP-1 contained in the Gln-MCP-1 preparation, and finally
converting the Gln-MCP-1 which is contained in the Gln-MCP-1 preparation or which was contained therein before the concentrating and/or purifying, into a pyroGlu-MCP-1 preparation which contains the protein molecule species pyroGlu-MCP-1, this step being carried out according to the “Process for preparing pyroGlu-MCP-1 from recombinantly produced Gln-MCP-1” as described above.

This process may optionally also include, for example, buffering the pyroGlu-MCP-1 preparation or further purification, e.g. by a subsequent step of column chromatography, dialysis, ultrafiltration, etc.

Methods of producing recombinant proteins by biotechnology are known. They comprise, in particular, fermentation, purification, concentration, and other steps. The step of conversion described above may be included at various points in a “multi-step process”, i.e. with an as yet largely unpurified or wholly purified MCP-1 protein solution as the starting solution. It is also conceivable, in particular, to have a process in which not yet (totally) converted Gln-MCP-1 in more, less or practically wholly purified form is lyophilised in order to improve its shelf life in as yet unconverted form and is in due course put back into solution and then converted into pyroGlu-MCP-1 according to the above process. Naturally, the protein solution obtained after conversion into pyroGlu-MCP-1 may also be lyophilised.

The product of the process, namely the pyroGlu-MCP-1 preparation subjected to N-terminal modification (conversion), contains according to the invention an amount of pyroGlu-MCP-1 of at least 90%, preferably at least 95% and more preferably at least 96%, based on the total content of MCP-1 (converted plus unconverted protein plus by-products formed by oxidation, for example).

The term (Gln- or pyroGlu-MCP-1-) preparation means that in the various stages of the process the MCP-1 protein is present in dissolved form in a (buffer) solution and hence other components may be present in addition to the MCP-1, i.e. in any case the ions of the buffer salt used and possibly also other salts, antioxidants, stabilisers, antimicrobial reagents, detergents, preservatives, complexing agents, etc.

According to a further aspect of the invention a composition is provided which contains pyroGlu-MCP-1 or a pyroGlu-MCP-1 preparation obtained by the processes described above, in which at least 90% of the MCP-1 contained in the composition (converted plus unconverted protein plus by-products; i.e. the “area under the curve” in an HPLC elution chromatograph, for example) are present in the form of the pyroGlu-MCP-1.

Thus, the invention also relates to an MCPO-1 preparation produced by the recombinant method particularly in prokaryotes and particularly preferably in E. coli wherein at least 90% of the MCP-1 protein is present as pyroGlu-MCP-1. When prepared in common expression cells the protein will frequently not exhibit the natural glycosylation pattern—unlike in native production. When prepared in prokaryotes such as e.g. E. coli, in particular, the pyroGlu-MCP-1 obtained or the pyroGlu-MCP-1 preparation obtained unlike the form which occurs in native expression in the human body will not be glycosylated and/or sialylated and will thus differ from a native protein of this kind or from a protein preparation obtained from a native source.

A composition of this kind according to the invention may be, in particular, a medicament or a pharmaceutical composition which contains, in addition to an amount of pyroGlu-MCP-1 which makes up at least 90%, 95% or 96% of the total MCP-1 content (converted plus unconverted protein plus by-products), conventional excipients and carriers, salts, antioxidants, stabilisers, antimicrobial reagents, detergents, preservatives, complexing agents, etc. The composition according to the invention may be used to treat patients suffering from an arterial occlusive disease such as in particular PAOD or CAD. The treatment comprises administering such a composition in a therapeutically effective amount by a suitable route, e.g. by intraarterial infusion or in the form of a periarterially deposited gel, from which the MCP-1 protein is released over a fairly long period.

Surprisingly it has also been found that, contrary to expectations, the incubation of a recombinantly prepared MCP-1 protein preparation in aqueous solution and at elevated temperature does not lead to its decomposition and biological inactivation; on the contrary, with a suitable choice of a number of parameters, this step, which is very unusual from a biotechnological point of view and is usually inherently undesirable, results in a surprisingly homogeneous, biologically fully active pyroGlu-MCP-1 preparation, which retains its homogeneity even during lengthy storage, i.e. is more stable than the Gln-MCP-1 (starting) preparation.

Bodies such as the Food and Drug Administration in the USA or the EMEA in Europe make the granting of marketing approval for a drug dependent on meeting numerous conditions which are imposed on the drugs manufacturers with the ultimate aim of protecting the patient. Thus, the manufacturer in question has to supply proofs which demonstrate, for example, the purity of the pharmaceutical product, its shelf life and its reproducibility of production. These three aspects are only a small selection, but play a central role precisely in the licensing of biopharmaceuticals, i.e. drugs which contain macromolecular active substances consisting of natural materials or derived therefrom (proteins, nucleic acids, proteoglycans, polysaccharides, etc.). Often, the requirements imposed on the (bio)pharmaceutical within the framework of this approval are very difficult to meet. For example, active substances based on a protein have to be subjected to intensive and complicated multi-stage purification after their production in a more or less complex cellular organism and must not be uncontrollably altered in structure, e.g. by oxidation or other spontaneous chemical reactions. Moreover, the end product should have a reasonable shelf life so that there is no need for complicated and expensive supply networks between the manufacturer and the prescribing doctor or patient or the clinic using the product—a requirement which is generally only met with great difficulty, particularly in the case of proteins, where it is important to maintain the correct secondary structure (folding).

In the case of the MCP-1 protein the quality of commercially obtainable preparations is frequently adequate in many respects for in vitro testing, for example. However, the inventors have found in the course of their work that the same preparations are by no means suitable for producing a drug which would be eligible for approval. Being left to stand in solution at ambient temperature even for a short time before being administered to the patient, which could not be ruled out in doctors' surgeries or hospitals, for example, leads to the occurrence of “breakdown” products in the MCP-1 preparations which were originally viewed as contamination, in the experience of the inventors. More intense purification of the Gln-MCP-1 originally provided as active substance briefly restored a high level of purity, but even this preparation was again unstable when briefly stored in solution and at ambient temperature. Operating a drug approval procedure on the basis of such an unstable active substance preparation is beset by tremendous problems, if not altogether hopeless.

Surprisingly, these problems can be overcome by the teaching of the invention, wherein an unusual and at first sight highly counter-productive step, namely incubation at elevated temperature (which was previously seen to positively promote inhomogeneity) was included in the actual preparation and purification process. In fact, when carried out under the conditions analysed in detail and perfected by the inventors, such incubation may bring about virtually quantitative conversion of the original active substance Gln-MCP-1 into the pyroGlu-MCP-1 form which was originally regarded as a contaminant. This latter form is then astonishingly stable against protein-denaturing influences such as incubation at elevated temperature and the like. Thus, an MCP-1 preparation is obtained which, by virtue of its high purity and homogeneity, exceptionally reproducible manufacture and stability on storage, is suitable for use as a pharmaceutical active substance which has good prospects of complying with the strict requirements in corresponding licensing procedures referred to earlier, and thus makes it possible to implement new therapeutic processes based on the activity of MCP-1.

According to a partial aspect of the invention a pyroGlu-MCP-1 preparation prepared by the process described above and in the claims is used
for the therapeutic treatment of vascular occlusive diseases such as, in particular, coronary artery disease (CAD), peripheral arterial occlusive disease (PAOD), cerebral and mesenterial arterial occlusive diseases, or
for preparing a pharmaceutical composition for the treatment of vascular occlusive diseases such as, in particular, coronary artery disease (CAD), peripheral arterial occlusive disease (PAOD), cerebral and mesenterial arterial occlusive diseases.

The invention thus also makes it possible to carry out a process for treating a patient suffering from the above-mentioned vascular occlusive diseases, wherein the pyroGlu-MCP-1 preparation prepared by conversion or the pharmaceutical composition prepared using a pyroGlu-MCP-1 preparation of this kind is administered by injection or infusion, for example.

With regard to the individual optimised process parameters it should also be noted that the biotechnologist will not easily be convinced that he or she should incubate a protein preparation for a fairly long period of, in some cases, several days at elevated temperatures of e.g. 60° C. Regarding the neutral to acidic pH of the incubating solution which was previously disclosed as optimum this is contrary to chemical logic: the conversion of glutamine to cyclic pyroglutamate is a nucleophilic substitution reaction in which a pair of free electrons acts on the attacking nitrogen atom of the C-alpha-amino group of the glutamate at the C atom of the C-gamma-amide group. An acidic pH would inherently lead to increased protonation of the C-alpha-amino group and would therefore have a negative effect on the speed of the reaction. However, this is precisely not the case, according to the inventors' findings. One possible explanation—which should not be interpreted restrictively—might be that the folded MCP-1 protein forms a micro-environment for the N terminus, in which in spite of the acidic pH of the surrounding aqueous solution the amino group is present in unprotonated form, i.e. a pair of free electrons is available for the nucleophilic attack.

### EXAMPLES

### Example 1

### Production of a pyroGlu-MCP-1 Preparation by the Process According to the Invention

Fermentation:

The fermentation was carried out with a strain Of Escherichia coli K12 (W3110). The strain was transformed with a ColE1 plasmid (pBR322 derivative) containing the following elements: the genomic sequence of hMCP-1 under the control of an all-purpose promoter (phosphatase, pPhoA), a ColE1-replication origin and the resistance gene for tetracycline. For fermentation the production strain was precultured in the shaking flask in LB medium containing tetracycline. Incubation was carried out at 37° C. until an OD of 1 was achieved. The preculture was transferred into the fermenter and further cultivated with stirring and with a supply of air at 37° C. The medium contained glucose, various salts, trace elements, yeast extract, amino acids and tetracycline. The pH was maintained at 6.8 with ammonia. As soon as the glucose put in had been used up the dissolved oxygen (pO₂) was kept constant at the intended level of 40% by the supply of glucose. The induction of the phosphatase promoter took place automatically as soon as the phosphate in the medium had been exhausted. After 39 hours the biomass was harvested with a tube centrifuge (CEPA) and stored at −70° C.

Cell Lysis and Protein Purification Steps:

The frozen cell pellet was resuspended in four times as much lysis buffer (200 mM Tris, 55 mM NaCl, 5 mM EDTA, pH 7.5) using an Ultraturrax. The cell lysis was carried out by two passages with a homogeniser at 460 bar. Cell fragments were removed with a CEPA centrifuge. The supernatant was optionally filtered with Polysep II (1.2 μm) filters (Millipore), and then loaded onto a column combination consisting of a Q sepharose FF and an SP sepharose FF. The columns were equilibrated with lysis buffer.

After the loading was complete the column combination was washed with lysis buffer and the Q sepharose column was clamped off. The SP sepharose was washed with lysis buffer, then with 5 M urea (in lysis buffer) and with lysis buffer again. The product was eluted in a linear NaCl gradient.

The eluate was salted with ammonium sulphate to a final concentration of 1.3 mol/L and centrifuged for 15 min at 3200 g. The supernatant was loaded onto a phenyl sepharose column which was equilibrated with 1.3 M ammonium sulphate (in lysis buffer).

The flow of phenyl sepharose was applied to a Source 30 RPC column which was equilibrated with lysis buffer. Washing was then carried out with lysis buffer, water and buffer A (5% EtOH, 0, 1% TFA). The product was eluted in a linear gradient from 20% buffer B (95% EtOH, 0.1% TFA) to 70% buffer B in 10 column volumes.

Conversion Step:

The eluate was diluted 1:10 in different buffers, e.g. phosphate buffer, 10-40 mM, pH 6.0-7.4. According to a preferred embodiment a value of between 6.0 and 6.5 was sought as the final pH of the conversion solution. The protein concentration was between 0.2 and 1.0 mg/mL.

The solution was filter sterilised and incubated at temperatures of 35-80° C. (in different batches) with gentle agitation (60 rpm). The progress of the reaction was monitored by HPLC analysis.

Final Purification Steps:

As soon as the end of the reaction was reached, the conversion solution was loaded onto an SP sepharose HP column which was equilibrated in buffer A (40 mM phosphate, pH 6.0). Elution was carried out with a linear NaCl gradient. The NaCl concentration in the eluate was about 350 mmol/L, and the protein concentration was about 3 mg/mL. The eluate was diluted with buffer A to a conductivity corresponding to 250 mM NaCl. Then it was further diluted with 40 mM phosphate, pH 6.0, 250 mM NaCl until the protein concentration was 1 mg/mL. The bulk solution thus formed was filter sterilised and stored at 4° C. or −70° C. until ready to be formulated.

Analysis:

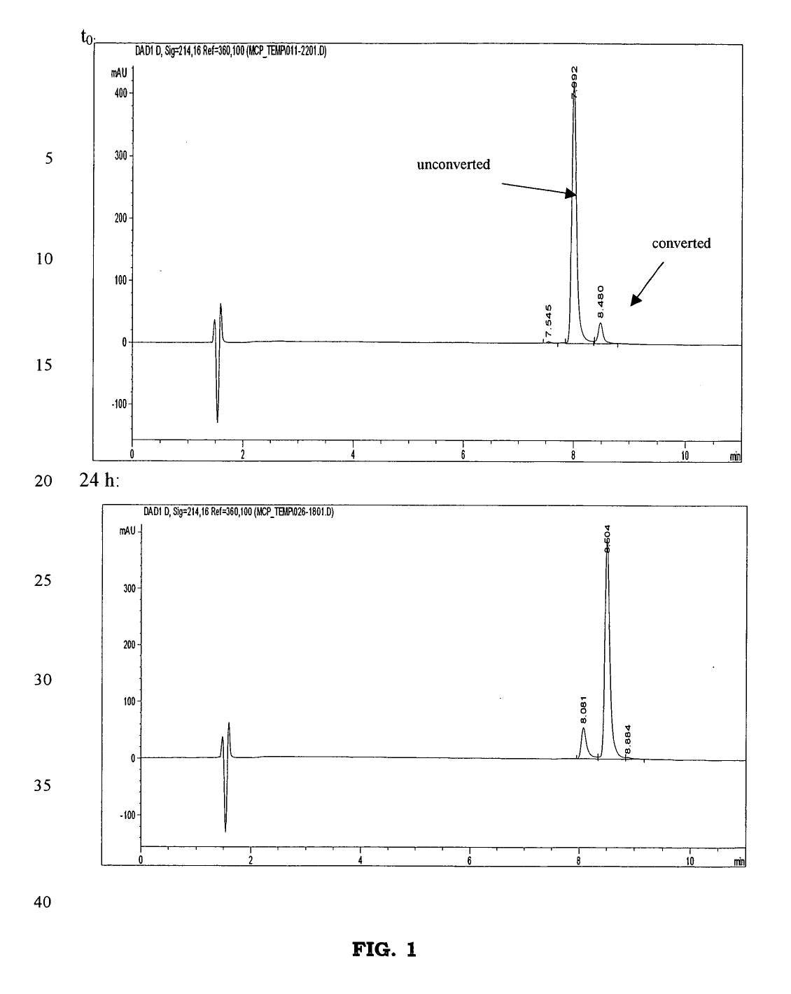

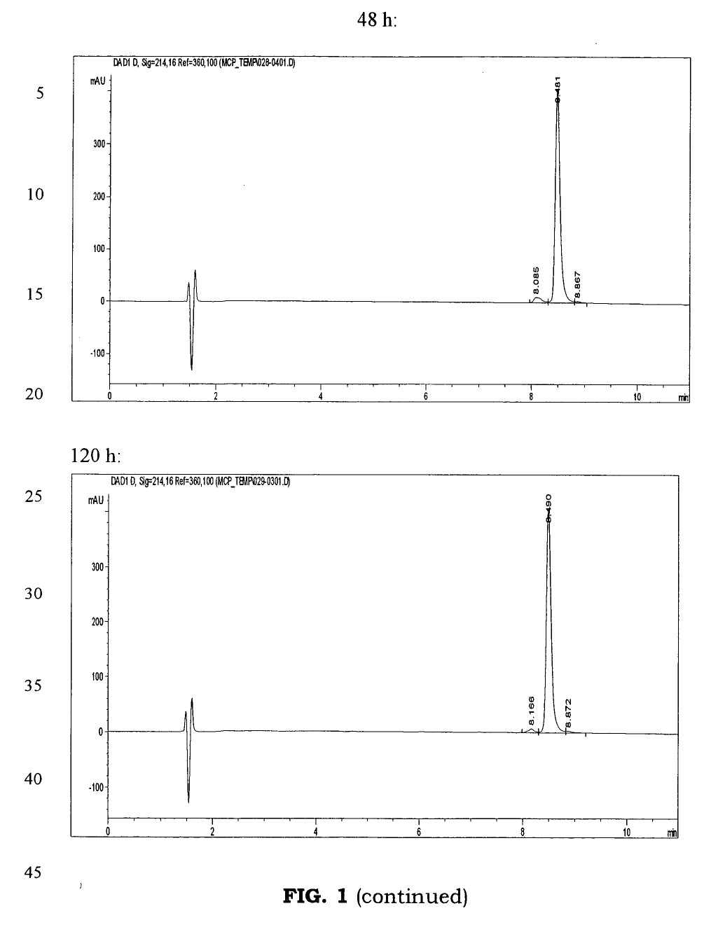

The progress of the conversion step discussed above was monitored by RP-HPLC. Chromatographs of the elution profiles were taken before the start of the conversion reaction (t₀) and at times t=24 h, t=48 h and t=120 h. As can be seen from the four chromatographs shown in FIG. 1, after 48 h approx. 90% of the protein had been converted into pyroGlu-MCP-1 at an incubation temperature of 35° C. HPLC analyses showed that the two disulphide bridges had formed properly.

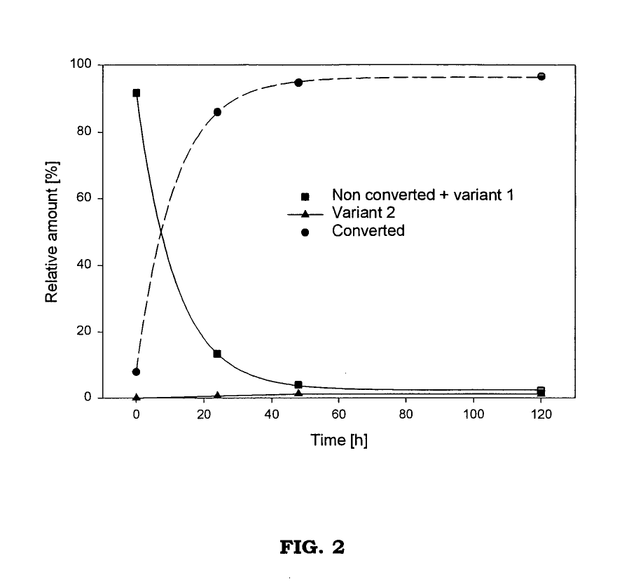

FIG. 2 shows the kinetics of the reaction of Gln-MCP-1 to pyroGlu-MCP-1 under the conditions described. It shows that as the duration of the reaction increases an impurity appears, which is referred to as “variant 2” in FIG. 2 and not further characterised.

Similar reaction patterns were observed when carrying out the conversion step in 0.1 M sodium acetate buffer, pH 5.5, or 0.1 M sodium citrate buffer, pH 3.5 (data not shown).

The reaction described above was repeated except that temperatures of 24° C., 60° C. and 80° C. were used instead of a reaction temperature of 35° C. As is clear from the data assembled in Table 3, no satisfactory yields of pyroGlu-MCP-1 were obtained at 24° C. within a reasonable time. At 60° C. and 80° C., on the other hand, significantly higher reaction rates are observed. The reaction rate at 80° C. was approximately 50 times the rate at ambient temperature.

TABLE 3

| | | speed | | | |
|---|---|---|---|---|---|
| | temperature | constant | half-life | | purity |
| | [° C.] | [1/h] | [h] | time | [%] |
| Comparison test 1: | 24 | 0.031 | 22.4 | 7.5 d | n.d. |
| 20 mM phosphate pH 6.0 | | | | | |
| 20 mM phosphate pH 6.0 | 35 | 0.089 | 7.8 | 2.6 d | 96.6 |
| 20 mM phosphate pH 6.0 | 60 | 0.660 | 1.1 | 8.8 h | 95.0 |
| 20 mM phosphate pH 6.0 | 80 | 1.670 | 0.4 | 3.2 h | 91.3 |
| 20 mM phosphate pH 6.0 | 35 | 0.041 | 16.9 | 5.6 d | n.d. |
| 150 mM NaCl | | | | | |
| 20 mM phosphate pH | 35 | 0.108 | 6.4 | 2.1 d | 95 |
| 6.0 5% EtOH | | | | | |
| Comparison test 2: | 35 | 0.037 | 18.7 | 6.2 d | n.d. |
| 20 mM phosphate pH 8.5 | | | | | |
| PBS Tween, 3.5% EtOH, | 35 | 0.040 | 17.3 | 5.7 d | 95 |
| 0.01% TFA pH 7.4 | | | | | |

n.d. = not determined, the end of the reaction had not been reached after the specified time

Certainly, a higher reaction temperature favours the formation of by-products or breakdown products, reducing the purity level—particularly when the test is carried out at 80° C. (Table 3).

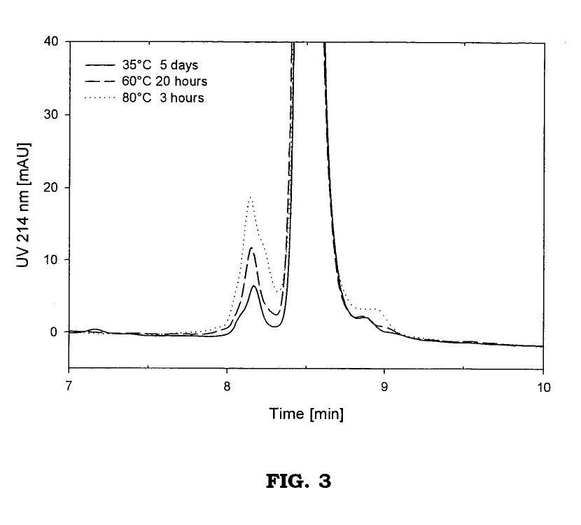

FIG. 3 shows in high resolution the chromatographs of the elution profile of pyroGlu-MCP-1 preparations which were obtained under the conditions summarised in Table 3 (20 mM phosphate buffer, 35° C., 60° C. or 80° C.). As can be seen from this, significantly more impurities occur with MCP-1 by-products or breakdown products at high temperatures, even with a short incubation period (e.g. 3 hours at 80° C.), than during five days' incubation at 35° C.

The content of (e.g. 5%) ethanol (EtOH) in certain embodiments of the process resulting from the production method did not have a negative effect on the reaction rates or purity of the product, under otherwise constant test conditions (20 mM phosphate buffer, pH 6.0).

### Example 2

### Production of a pyroGlu-MCP-1 Preparation by an Alternative Process According to the Invention

The fermentation, cell lysis and protein purification steps were carried out as explained in Example 1.

Conversion Step:

The eluate of the Source 30 RPC was diluted 1:10 with PBS, 0.02% Tween 20. The protein concentration was then between about 0.2 and 0.5 mg/mL, the pH was about 7.4. In addition to the buffer components the solution contained about 3.5% EtOH and 0.01% TFA, which originated from the eluate of the previous Source 30 RPC step. The solution was filter sterilised into polypropylene flasks with a Millipak 20 (0.2 μm, Millipore) and incubated at 35° C. with gentle agitation. The progress of the reaction was monitored by HPLC analysis. As soon as the end of the reaction was reached the conversion solution was ultradiafiltered with a Pellikon 2 UDF membrane (3 k, Millipore) against PBS, 0.02% Tween 20, and the protein concentration was adjusted to 0.1 mg/mL. The preparation was decanted in 1 mL batches into glass containers through a Millipore GV filter (0.22 μm) under sterile conditions.

Analysis:

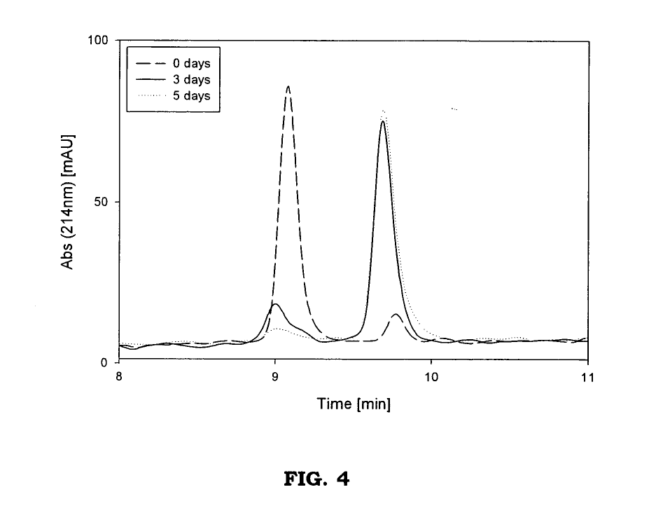

The progress of the conversion step was monitored by RP-HPLC. As can be seen in FIG. 4, after 5 days more than 90% of the protein had been converted into pyroGlu-MCP-1, while surprisingly in spite of the long incubation period a very high purity of 95% was obtained (Table 3). As is apparent from a comparison of mixtures containing 20 mM phosphate buffer and those containing 20 mM phosphate buffer plus 150 mM salt (Example 1, Table 3, line 3 to line 6), the relatively low speed constant in mixtures containing PBS as incubation solution may be put down to the higher salt concentration, ionic strength or osmolarity.

### Example 3

### Preparation of a Pharmaceutical Composition Based on the MCP-1 Preparation Produced According to Example 1 or Example 2

The pyroGlu-MCP-1 preparation obtained in Example 1 or Example 2 was diluted to a concentration of 0.1 mg/mL in PBS (sodium chloride, disodium hydrogen phosphate, potassium chloride, potassium dihydrogen phosphate; pH 7.0), also containing 0.02% Tween 20, and transferred into glass containers in volumes of 1 mL. The finished pharmaceutical solution was clear, colourless and odourless and can be administered by injection or infusion.

### Example 4

### Comparison of the Biological Activity of pyroGlu-MCP-1 and Gln-MCP-1

Measuring Principle:

MCP-1 binds to and activates the MCP-1 receptor CCR2b. The activation of the receptor leads to an influx of calcium into the cytosol. This can be measured using a Fluorescence Imaging Plate Reader (FLIPR; Molecular Devices). To do this, the inactive fluorescent dye ester Fluo-4 AM is sluiced into cells which express hCCR2b on their surface, this ester then being cleaved by intracellular esterases. In this form the fluorescent dye binds Ca²⁺ ions. On excitation with a wavelength of 488 nm there is an emission with a peak at 528 nm. The intensity of the emission is dependent on the concentration of calcium in the cytosol. The change in intensity of the emitted light thus correlates with the concentration of calcium in the cytosol which is in turn dependent on the state of activation of the receptor.

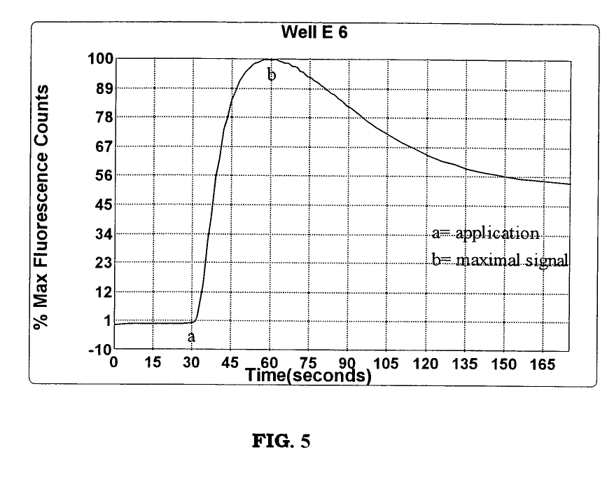

A characteristic time-triggered measuring signal after the addition of MCP-1 is shown in FIG. 5. It is apparent from this that after the addition of MCP-1 there is a rapid release of calcium which is linked with a sharp rise in fluorescence. The peak (b) is reached just a few seconds after the application (a). The interval between the application (a) and maximum stimulation (b) is roughly 20-30 sec.

In the tests that follow, the evaluation was carried out using the maximum values as they are less affected by secondary effects such as e.g. a calcium-induced calcium influx, and therefore more precise measurement is possible.

Preparation of Stably Expressing CHO/hCCR2B-K1 Cells:

The coding region of the human CCR2b receptor (Gene bank Accession No: D29984) was amplified by PCR. Then the 1.08 kb BamHI-XbaI fragment was cloned into an expression vector. CHO-K1 cells were transfected with this plasmid acting as an expression vector.

The cells were cultivated in a culture medium based on Ham's F12 medium and regularly passaged. On the day before the measurement 5000 cells were plated out in 384-well assay plates (Corning Costar) with 40 μl of culture medium and left to adhere overnight (approx. 24 h) at 37° C., 5% CO₂, 95% relative humidity. In all the assays the measurements were carried out four times.

On the day of the test, first of all the substance plates were prepared. Hanks buffer with 0.1% BSA (protease-free) was used as the diluting buffer for the various MCP-1 preparations. Hanks buffer with 0.1% BSA was used as the blank control. Then 40 μl/well of Fluo-4 dye medium were added to the cells and the preparations were incubated for 45 min. at 37° C., 5% CO₂, 95% relative humidity. The stained cells were then washed four times with 60 μl washing buffer, leaving a residue of 25 μl of washing buffer in each well. The cells were then incubated with washing buffer for a further 5 min at ambient temperature.

To measure the fluorescence the FLIPR measuring device was adjusted so that stained and unstained wells differed by at least a factor 1:5 and the stained wells had approx. 11,000 fluorescence counts. The other FLIPR settings were:

| | |
|---|---|
| Excitation: | 488 nm |
| Emission: | 510-570 nm (band pass) |
| Negative correction: | Hanks/BSA (= comparison with blank control) |
| Bias subtraction: | 6 (= levelling all the wells to 0 before adding the |
| | substance) |
| Presoak of the tips: | with 25 μl of the relevant substance solution from |
| | the substance plate (= minimising an adhesion |
| | artefact) |

The measuring process comprised the following steps:
6 intervals in a 5 sec. cycle
15 μl substance added
60 intervals in a 1 sec. cycle
18 intervals in a 5 sec cycle

For evaluation the maximum signal of the 78 intervals after the addition of the substance was used. The control mixtures used were:

| | |
|---|---|
| blank control: | for negative correction |
| positive control (ATP): | for checking the staining |
| reference control (standard): | monitoring the receptor expression on |
| | the cells |
| | basis of calculation for determining the |
| | activity of unknown samples |

The samples to be tested were pipetted parallel with the reference control in identical dilution steps. The concentrations used in the tests were selected so as to be in the EC₅₀ region of the reference control.

Comparison of MCP-1 Preparations According to the Invention and not According to the Invention:

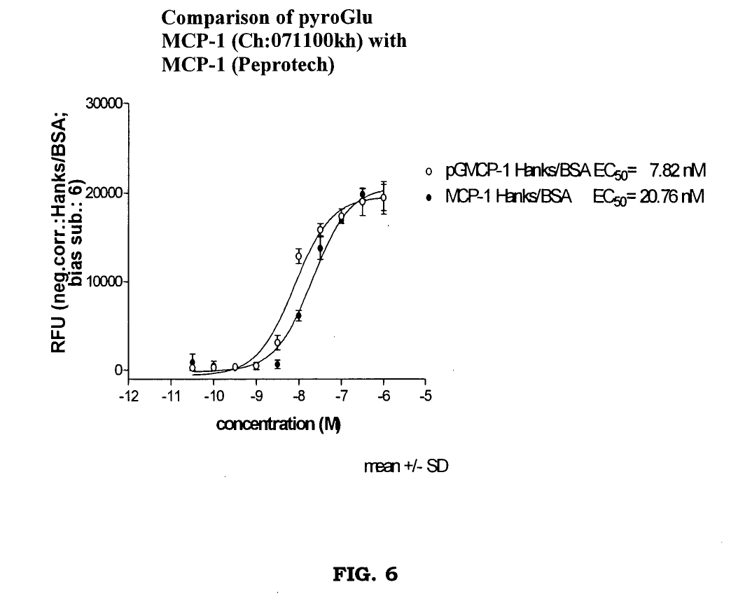

Two pyroGlu-MCP-1 preparations (“Batch 071100kh” and “Batch 0141921”) prepared by the process according to the invention and an MCP-1 preparation prepared by the recombinant method, whose N-terminal glutamine group had not been converted into a pyroglutamate group in the process step according to the invention as described above (MCP-1 preparations obtainable from Peprotech) were tested and compared using the test system described previously. The EC₅₀ was determined from the measurement curves recorded as described above (see FIG. 6).

Virtually identical EC₅₀ values were obtained for the two pyroGlu-MCP-1 preparations, while the values for the MCP-1 preparation not according to the invention and not N-terminally modified differed significantly (FIG. 6). The EC₅₀ value of the latter preparation proved to be worse by a factor 2 to 3 (pyroGlu-MCP-1: EC₅₀=7.82 nM; MCP-1 preparation from Peprotech: EC₅₀=20.76 nM).

The Biological Activity of the Two Preparations was Calculated as Follows:

First, a positive correction is made to the pyroGlu MCP-1 at the concentration 1e-8 M, i.e. the value obtained at a concentration of 10 nM pyroGlu-MCP-1 was set at 100%. This is in the almost linear part of the ascent of the curve. Using this percentage signal the activity of other preparations, such as e.g. the Peprotech MCP-1 is calculated according to the formula
% activity₍ₛₐₘₚₗₑ₎=RFU_{(sample at 1e-8M)}×100%/RFU_{(standard at 1e-8M)}

In the above Example, as shown in Table 4, this yields a value of 48% activity for the non-N-terminally modified MCP-1 preparation based on the pyroGlu-MCP-1 preparation.

TABLE 4
measured valu(neg .corr.:Hanks/BSA; bias substraction:6)

| | value 1 | value 2 | value 3 | value 4 |
|---|---|---|---|---|
| pyroGlu MCP-1 | 12137.63 | 13919.36 | 12286.6 | 13249.68 |
| (071100kh) at 1e−8M | | | | |
| MCP-1 (Peprootech) at | 6216.81 | 6125.89 | 6836.11 | 5425.31 |
| 1e−=8M | | | | |

| | average | SD |
|---|---|---|
| pyroGlu MCP-1 | 1289.3 | 727.8 |
| (071100kh) at 1e−8M | | |
| MCP-1 (Peprootech) at | 6151.0 | 500.2 |
| 1e−8M | | |

% activity _{(MCP-1 (Peprotech))} = 616151 × 100% / 12898.3 = 48% activity

### Example 5

### Temperature Sensitivity of pyroGlu-MCP1

pyroGlu-MCP-1 was incubated for 1 or 2 hours at 56° C. or 95° C. The biological activity still present thereafter was then measured using the test system in Example 4.

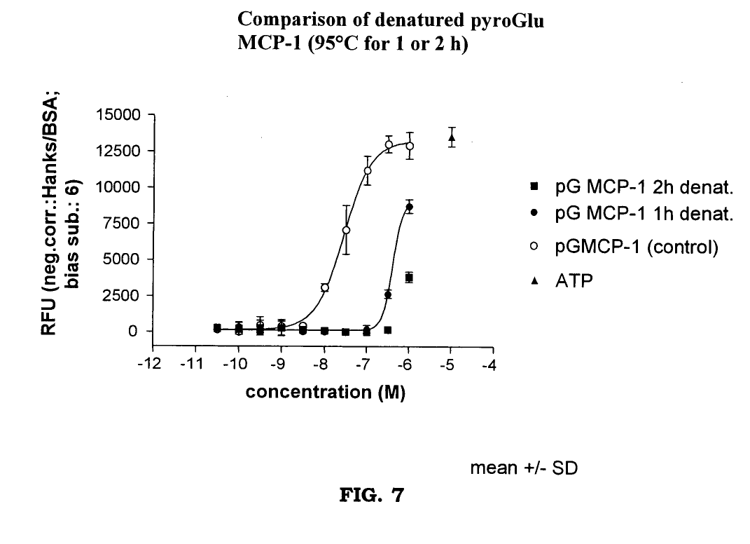

It was found that no denaturing occurs when a pyroGlu-MCP-1 preparation is incubated at 56° C. for either one hour or two hours. However, as shown in FIG. 7, incubation at 95° C. results in a very marked denaturing effect.

### Example 6

### Treatment of Patients Suffering from an Arterial Occlusive Disease:

For treating PAOD patients a pyroGlu-MCP-1 preparation as prepared above is adjusted to a concentration of between 1.2 and 120 μg/ml. Immediately before use this solution is adjusted to a final concentration of between 0.1 and 10 μg/ml and infused into the patient at a flow rate of 2 to 12 ml/min over a period of 1 to 6 hours by intraarterial route close to the region affected by the vascular occlusion. The infusion may be repeated after 1 to 7 days.

1. Process for preparing pyroGlu-MCP-1 from recombinantly produced Gln-MCP-1, wherein Gln-MCP-1 is incubated
at a temperature in the range from 30° C. and 80° C.
in a buffer solution with
a salt concentration in the range from 10 mM to 160 mM and
at a pH in the range from 2 to 7.5
until at least 90% of the MCP-1 is present in the form of the pyroGlu-MCP-1.

2. Process according to claim 1, wherein the buffer solution is a phosphate buffer with a concentration in the range from 20 mM to 50 mM and with a pH in the range from 3.5 to 6.5.

3. Process according to one of claims 1 or 2, wherein the buffer solution additionally contains a detergent, an antioxidant, a preservative, a stabiliser, an antimicrobial reagent and/or a complexing agent.

4. Process for preparing a pyroGlu-MCP-1 preparation, comprising at least the steps of
preparing a Gln-MCP-1 preparation by expression of a gene construct coding for MCP-1 in a host cell,
optionally concentrating and/or purifying the Gln-MCP-1 contained in the Gln-MCP-1 preparation,
converting the Gln-MCP-1 of the Gln-MCP-1 preparation into a pyroGlu-MCP-1 preparation which contains pyroGlu-MCP-1, according to one of processes 1 to 3, and
optionally buffering and/or further purifying the pyroGlu-MCP-1 preparation,
the proportion of pyroGlu-MCP-1 based on the total content of MCP-1 in the resulting pyroGlu-MCP-1 preparation being at least 90%.

5. Composition containing a pyroGlu-MCP-1 preparation prepared according to claim 4, wherein at least 90% of the MCP-1 contained in the pyroGlu-MCP-1 preparation is present in the form of the pyroGlu-MCP-1.

6. Process for preparing a pharmaceutical composition containing pyroGlu-MCP-1, wherein a pyroGlu-MCP-1 preparation prepared according to claim 4 is used.

7. Medicament or pharmaceutical composition containing a pyroGlu-MCP-1 preparation prepared according to claim 4, wherein at least 90% of the MCP-1 contained therein is in the form of the pyroGlu-MCP-1.

8. Medicament or pharmaceutical composition according to claim 7, containing pyroGlu-MCP-1 in a phosphate buffer with sodium chloride and optionally a detergent as additives.

9. Use of pyroGlu-MCP-1 or a pyroGlu-MCP-1 preparation prepared according to claim 4 for preparing a pharmaceutical composition for the treatment of vascular occlusive diseases such as, in particular, coronary artery disease (CAD), peripheral arterial occlusive disease (PAOD), cerebral and mesenterial arterial occlusive diseases.

10. Recombinant MCP-1 preparation produced by the process according to claim 4, the biological activity of which, with respect to a recombinantly produced MCP-1 preparation which has not been subjected to a process according to claim 1 (N-terminally unmodified MCP-1 preparation), is in the ratio 100:48 or higher.

11. Recombinantly produced MCP-1 preparation, wherein at least 90% of the MCP-1 protein is present as pyroGlu-MCP-1.

12. MCP-1 preparation according to claim 11, wherein the pyroGlu-MCP-1 is present in non-glycosylated form.

13. Process for inducing a biological or physiological reaction which substitutes for or potentiates the biological or physiological activity of endogenous native MCP-1-protein, characterised in that a composition according to claim 5 or a pharmaceutical composition according to claim 7 or a preparation according to at least one of claims 10 to 12 is added to cells or tissues or organs in an amount which is suitable for evoking the biological or physiological activity.

14. Process according to claim 13, wherein the cells or tissues or organs comprise CCR-2 and/or CCR-4-receptors.

15. Process according to claim 14, wherein the cells are mammalian cells which natively or recombinantly express the MCP-1 receptor subtype CCR2, particularly CCR2b.
